(19) United States
(12) Patent Application Publication
(10) Pub. No.: US 2014/0012232 A1
(43) Pub. Date: Jan. 9, 2014

(54) PRODUCTION METHOD OF MEDICAL WIRE AND MEDICAL WIRE

(71) Applicant: Olympus Corporation, Tokyo (JP)

(72) Inventor: TOMONORI IMAMURA, Tokyo (JP)

(73) Assignee: Olympus Corporation, Tokyo (JP)

(21) Appl. No.: 14/022,607

(22) Filed: Sep. 10, 2013

Related U.S. Application Data
(63) Continuation of application No. PCT/JP2012/064836, filed on Jun. 8, 2012.

(30) Foreign Application Priority Data
Jun. 10, 2011 (JP) 2011-130124

(51) Int. Cl. A61M 25/09

(52) U.S. Cl. CPC A61M 25/09
USPC 604/528; 219/121.64

(57) ABSTRACT

A production method of a medical wire includes a melting portion forming process that forms an massive melting portion in a wire end by melting and solidifying the wire end of a wire; and a joining process that contacts the melting portion with a joined part, and melts and solidifies the melting portion and the joined part to form a fusion portion, thereby joining the wire end and a joined part via a fusion portion.

This application claims priority to and the benefit of Japanese Patent Application No. 2011-130124 filed on Jun. 10, 2011, and is a continuous application of international patent application No. PCT/JP2012/064836 filed on Jun. 8, 2012, the disclosures of which are incorporated herein by reference.

### BACKGROUND OF THE INVENTION

1. Field of the Invention

The present invention relates to a medical wire and a production method of a medical wire.

2. Description of Related Art

In the past, medical wires used for medical care, for example, medical wires used in a catheter, a treatment tool, an endoscope or the like, include along portion and a distal end portion. In some cases, in order to add functions such as treatment or to form a loop shape, the distal end portion of the medical wire of the related art includes a configuration in which a plurality of wires are joined. In a medical wire, a stranded wire in which a plurality of thin wires are twisted and a single line wire have been used in combination in order to obtain excellent flexibility.

As a production method of the medical wire, there is a method of joining the end portions of the plurality of wires with respect to a connection fitting through welding, soldering, or pressure bonding.

For example, Japanese Patent No. 3182441 discloses a method of joining the stranded wires by inserting the end portions of the stranded wires used as a manipulation wire of an endoscope through a coupling pipe member in an abutted state to perform laser-welding on the end portions of each stranded wire to the coupling pipe member.

Furthermore, Japanese Patent No. 4494782 discloses a method includes steps of forming end portions of a guide wire used in a blood vessel in a tapered shape, abutting the end portions of a guide wire with each other, joining an outer circumferential portion thereof by a tube-shaped connector formed of a metal, or the like, and grinding an external shape of the tube-shaped connector to be adjusted to an outer diameter of the guide wire.

### SUMMARY OF THE INVENTION

According to a first aspect of the invention, a production method of a medical wire includes a melting portion forming process that forms a massive melting portion in a wire end by melting and solidifying the wire end; and a joining process that contacts the melting portion with a joined part, and melts and solidifies the melting portion and the joined part to form a fusion portion, thereby joining the wire end and a joined part via a fusion portion.

According to a second aspect of the invention, in the production method of the medical wire according to the first aspect, the wire may include a stranded wire.

According to a third aspect of the invention, in the production method of the medical wire according to the first or second aspect, the melting portion may be formed in a substantially spherical shape in the wire end.

According to a fourth aspect of the invention in the production method of the medical wire according to any one of the first to third aspects, the melting portion may be formed by irradiating the wire end and the joined part with a laser beam.

According to a fifth aspect of the invention, in the production method of the medical wire according to any one of the first fourth aspects, the melting portion may be formed in a first wire, and the first wire or a second wire that is different from the first wire may have the joined part.

According to a sixth aspect of the invention, in the production method of the medical wire according to the fifth aspect, the joined part may be a wire end different from the wire end formed with the melting portion of the first wire or a wire end of the second wire.

According to a seventh aspect of the invention, in the production method of the medical wire according to the sixth aspect, the fusion portion may be formed in a rod shape and have an outer diameter that is less than or equal to the maximum outer diameter of outer diameters of the first wire and the second wire.

According to an eighth aspect of the invention, in the production method of the medical wire according to the fifth aspect, the joined part may be an outer circumferential portion of a certain position of one of the first wire and the second wire.

According to a ninth aspect of the invention, a medical wire includes a fusion portion in which materials of a wire end of a wire and a joined part are melted and fused, and the fusion portion is formed and joined between the wire end and the joined part that is to be joined with the wire end.

According to a tenth aspect of the invention, in the medical wire according to the ninth aspect, the wire may include a stranded wire.

According to an eleventh aspect of the invention, in the medical wire according to the ninth or tenth aspect, the joined part may be a wire end that is different from the wire end formed with the melting portion of the wire.

According to a twelfth aspect of the invention, in the medical wire according to the eleventh aspect, the fusion portion may be formed in a rod shape having an outer diameter that is less than or equal to the maximum outer diameter of outer diameter of the wire.

According to a thirteenth aspect of the invention, in the medical wire according to the ninth or tenth aspect, the joined part may be an outer circumferential portion of the wire.

### BRIEF DESCRIPTION OF THE DRAWINGS

FIG. 1A is a schematic front view that illustrates a schematic configuration of a medical wire according to a first embodiment of the invention.

FIG. 1B is a cross-sectional view taken along line A-A of FIG. 1A.

FIG. 1C is a cross-sectional view taken along line B-B of FIG. 1A.

FIG. 1D is a cross-sectional view taken along line C-C of FIG. 1A.

FIG. 2A is a schematic process explanatory drawing that illustrates a melting portion forming process of the production method of the medical wire according to the first embodiment of the invention.

FIG. 2B is a schematic process explanatory drawing that illustrates a melting portion forming process of the production method of the medical wire according to the first embodiment of the invention.

FIG. 3 is a photographic image that illustrates an example of the melting portion formed in the melting portion forming process of the production method of the medical wire according to the first embodiment of the invention.

FIG. 4 is a graph that illustrates an example of a test result illustrating a relationship between a laser output and an outer diameter of the melting portion.

FIG. 5 is a photographic image that illustrates an example of a form of an end portion of the wire when the laser output is too small.

FIG. 6A is a schematic process explanatory drawing that illustrates a joining process of the production method of the medical wire according to the first embodiment of the invention.

FIG. 6B is a schematic process explanatory drawing that illustrates the joining process of the production method of the medical wire according to the first embodiment of the invention.

FIG. 7A is a photographic image that illustrates an example of the wire before joining in the joining process of the production method of the medical wire according to the first embodiment of the invention.

FIG. 7B is a photographic image that illustrates an example of the wire after joining in the joining process of the production method of the medical wire according to the first embodiment of the invention.

FIG. 8A is a photographic image that illustrates an example of the wire before joining in the joining process of a comparative example.

FIG. 8B is a photographic image that illustrates an example of the wire after joining in the joining process of a comparative example.

FIG. 9A is a schematic front view that illustrates a schematic configuration of the medical wire according to a first modified example of the first embodiment of the invention.

FIG. 9B is a cross-sectional view taken along line D-D of FIG. 9A.

FIG. 9C is a cross-sectional view taken along line E-E of FIG. 9A.

FIG. 9D is a cross-sectional view taken along line F-F of FIG. 9A.

FIG. 10A is a schematic process explanatory drawing that illustrates a melting portion forming process of the production method of the medical wire according to the first modified example of the first embodiment of the invention.

FIG. 10B is a schematic process explanatory drawing that illustrates a melting portion forming process of the production method of the medical wire according to the first modified example of the first embodiment of the invention.

FIG. 11A is a photographic image that illustrates an example of the wire before joining in the joining process of the production method of the medical wire according to the first modified example of the first embodiment of the invention.

FIG. 11B is a photographic image that illustrates an example of the wire after joining in the joining process of the production method of the medical wire according to the first modified example of the first embodiment of the invention.

FIG. 12 is a schematic front view that illustrates a schematic configuration of a medical wire according to a second modified example of the first embodiment of the invention.

FIG. 13A is a schematic front view that illustrates a schematic configuration of a medical wire according to a third modified example of the first embodiment of the invention.

FIG. 13B is a cross-sectional view taken along line G-G of FIG. 13A.

FIG. 13C is across-sectional view taken along line H-H of FIG. 13A.

FIG. 13D is a cross-sectional view taken along line J-J of FIG. 13A.

FIG. 14A is a schematic process explanatory drawing that illustrates the joining process of the production method of the medical wire according to the third modified example of the first embodiment of the invention.

FIG. 14B is a schematic process explanatory drawing that illustrates the joining process of the production method of the medical wire according to the third modified example of the first embodiment of the invention.

FIG. 15A is a schematic front view that illustrates a schematic configuration of a medical wire according to a fourth modified example of the first embodiment of the invention.

FIG. 15B is a schematic front view that illustrates a schematic configuration of a medical wire according to a fifth modified example of the first embodiment of the invention.

FIG. 15C is a schematic front view that illustrates a schematic configuration of a medical wire according to a sixth modified example of the first embodiment of the invention.

FIG. 16A is a schematic process explanatory drawing that illustrates a joining process of a production method of a medical wire according to a seventh modified example of the first embodiment of the invention.

FIG. 16B is a schematic process explanatory drawing that illustrates the joining process of the production method of the medical wire according to the seventh modified example of the first embodiment of the invention.

FIG. 17A is a front view that illustrates an example of a wire before joining in a joining process of a production method of a medical wire according to a second embodiment of the invention.

FIG. 17B is a front view that illustrates an example of the wire after joining in the joining process of the production method of the medical wire according to the second embodiment of the invention.

FIG. 18A is a front view that illustrates an example of the wire before joining in a joining process of a production method of a medical wire according to a modified example of the second embodiment of the invention.

FIG. 18B is a front view that illustrates an example of the wire after joining in the joining process of the production method of the medical wire according to the modified example of the second embodiment of the invention.

### DETAILED DESCRIPTION OF THE INVENTION

Hereinafter, embodiments of the invention will be described with reference to the attached drawings.

### First Embodiment

A medical wire according to a first embodiment of the invention will be described.

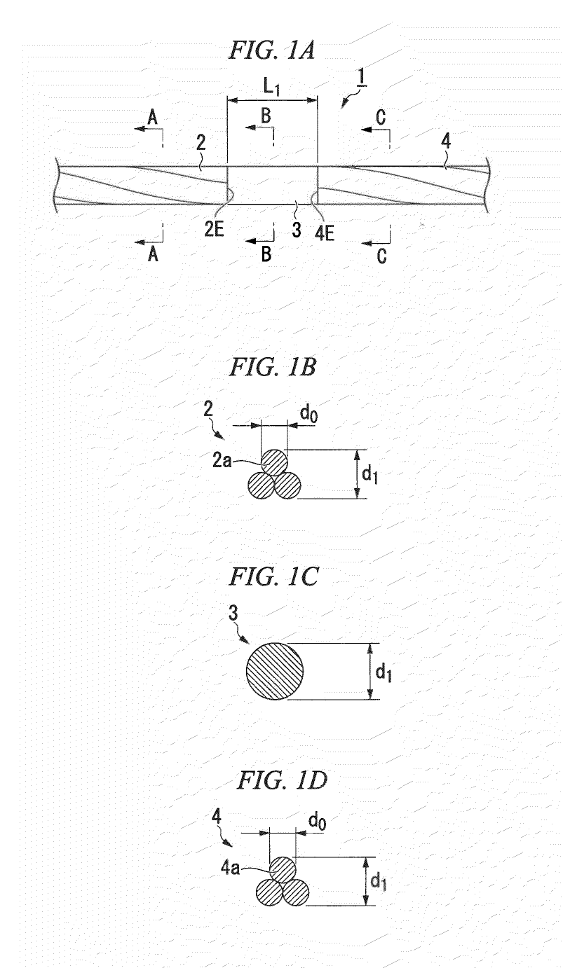

FIG. 1A is a schematic front view that illustrates a schematic configuration of a medical wire according to a first embodiment of the invention. FIG. 1B is a cross-sectional view taken along line A-A of FIG. 1A. FIG. 1C is a cross-sectional view taken along line B-B of FIG. 1A. FIG. 1D is a cross-sectional view taken along line C-C of FIG. 1A.

As illustrated in FIG. 1A, a joining wire 1 of the embodiment is a medical wire in which stranded wire portions 2 and 4 are joined via a fusion portion 3 in each of wire ends 2E and 4E.

The stranded wire portion 2 (first wire) is a linear member that is formed by twisting a plurality of element wires. In the stranded wire portion 2, a suitable configuration of the stranded wire may be adopted. For example, various wire configurations such as “1×3” in which three element wires are twisted to form one stranded wire, or “1×19” in which nineteen element wires are twisted to form one stranded wire may be adopted.

In the joining wire 1 of the embodiment, as an example, as illustrated in FIG. 1B, the stranded wire portion 2 has a wire configuration of 1×3. That is, the stranded wire portion 2 has a configuration in which three element wires 2a having an element wire diameter d₀ are twisted. For this reason, a wire outer diameter d₁ has a relationship of d₁=2·d₀. A direction in which the stranded wire 2 is twisted is not particularly limited.

As a material of the element wire 2a, a suitable material for metallic wire can be adopted depending on applications. For example, stainless steel, an iron-based alloy, a copper-based alloy, an aluminum-based alloy, a nickel-titanium-based alloy, a titanium-based alloy, a cobalt-based alloy or the like, or a configuration in which a plurality of materials of these materials are combined may be adopted. In the embodiment, since the stranded wire portion 2 performs the treatment of the distal end, SUS316 having high corrosion-resistance and acid-resistance even among stainless steels is adopted.

The stranded wire portion 4 (second wire) is able to adopt the same wire configuration, the same element wire diameter and the same element wire material as the stranded wire 2. In the embodiment, the stranded wire portion 4 has the same wire configuration and the same element wire diameter as the stranded wire portion 2, but is different from the stranded wire portion 2 only in the material of the element wire.

That is, as illustrated in FIG. 1D, the stranded wire portion 4 has a configuration of a wire outer diameter d₁ in which three element wires 4a having an element wire diameter d₀ are twisted. As the material of the element wire 4a, common SUS304 is adopted.

For this reason, in the stranded wire portions 2 and 4, although the wire configurations and the wire outer diameters are equal, corrosion-resistance and acid-resistance are different due to the change of the materials of the wires.

The fusion portion 3 is a part in which the end portion (wire end) of the stranded wire portion 2 and the end portion (wire end, joined part) of the stranded wire portion 4 are melted, mixed and solidified. In the embodiment, as illustrated in FIG. 1C, the fusion portion 3 is formed in a solid rod shape having a substantially circular cross-section with an outer diameter of d₁ or less. As illustrated in FIG. 1A, a length of the fusion portion 3 in an axial direction is L₁. The end portion of the fusion portion 3 in the axial direction is connected to the wire ends 2E and 4E.

That is, the fusion portion 3 has a shape which is a cylindrical rod shape having the outer diameter of d or a rod shape (hereinafter referred to as a “medium-fine rod shape”) in which an axial cross-sectional diameter is d₁ in the end portion and becomes gradually thinner toward an intermediate portion. When the axial cross-sectional diameter becomes thinner in the intermediate portion, a minimum cross-sectional diameter is set to a size of an extent in which tensile strength and bending strength become a permissible range in use.

For example, the joining wire 1 having such a configuration can be used as a treatment tool, a manipulation wire of an endoscope and the like.

Furthermore, for example, the joining wire 1 can be used as a wire that configures a treatment tool portion such as a snare loop and a blade portion, in treatment tools such as a snare and a high-frequency knife portion. Furthermore, the stranded wire portion 2 can also be used as the treatment tool portion, and the stranded wire portion 4 can also be used as the manipulation wire.

Next, a production method of the medical wire of the embodiment for producing the joining wire 1 having such a configuration will be described.

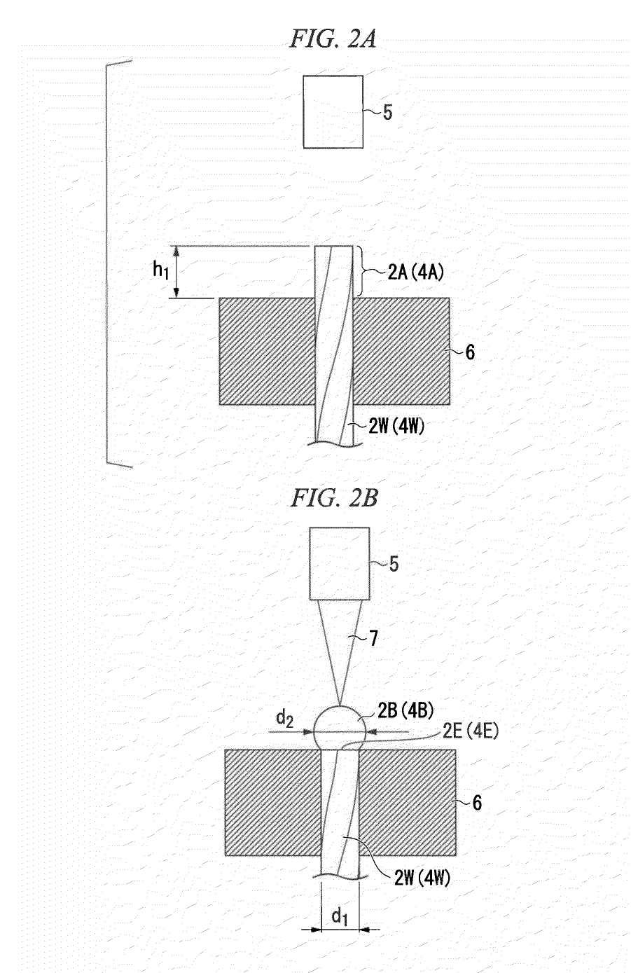

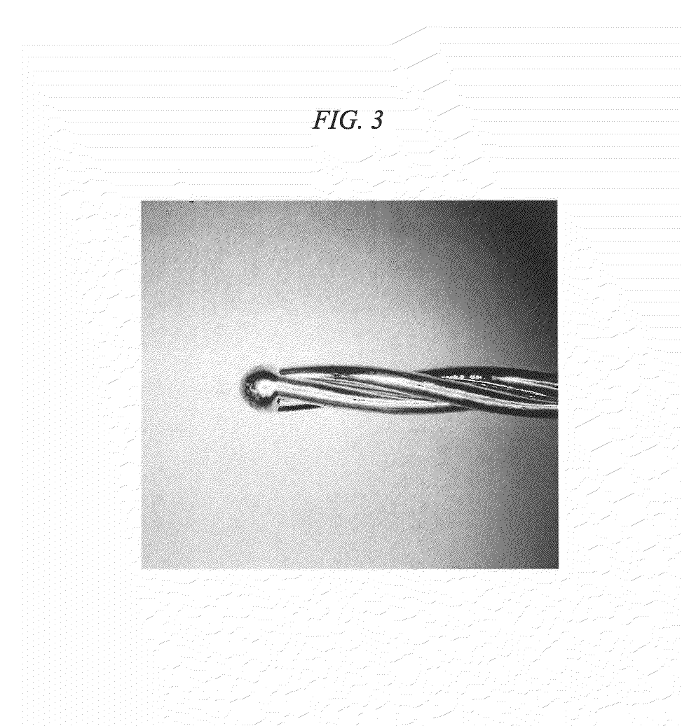

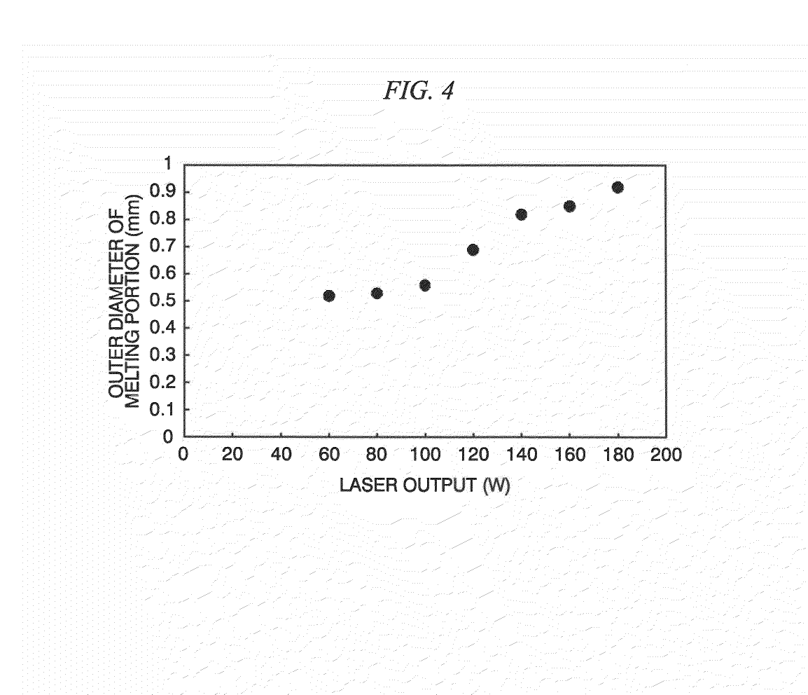

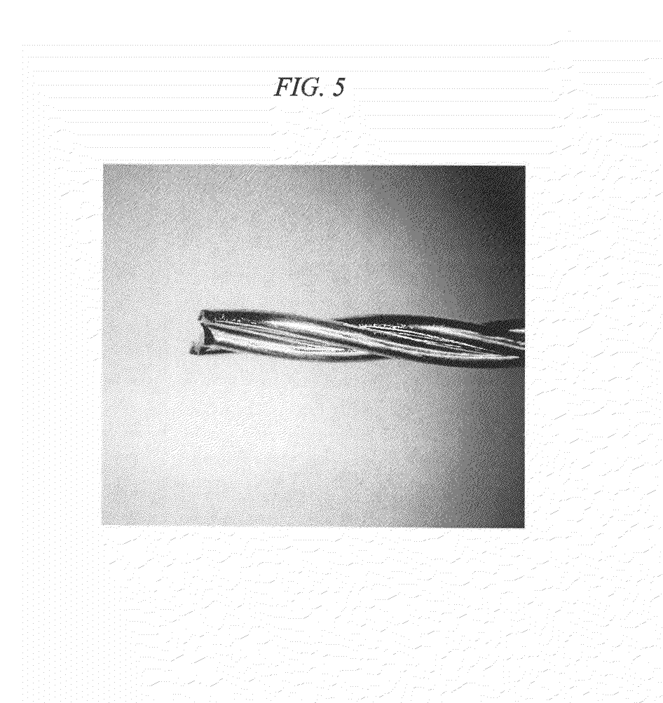

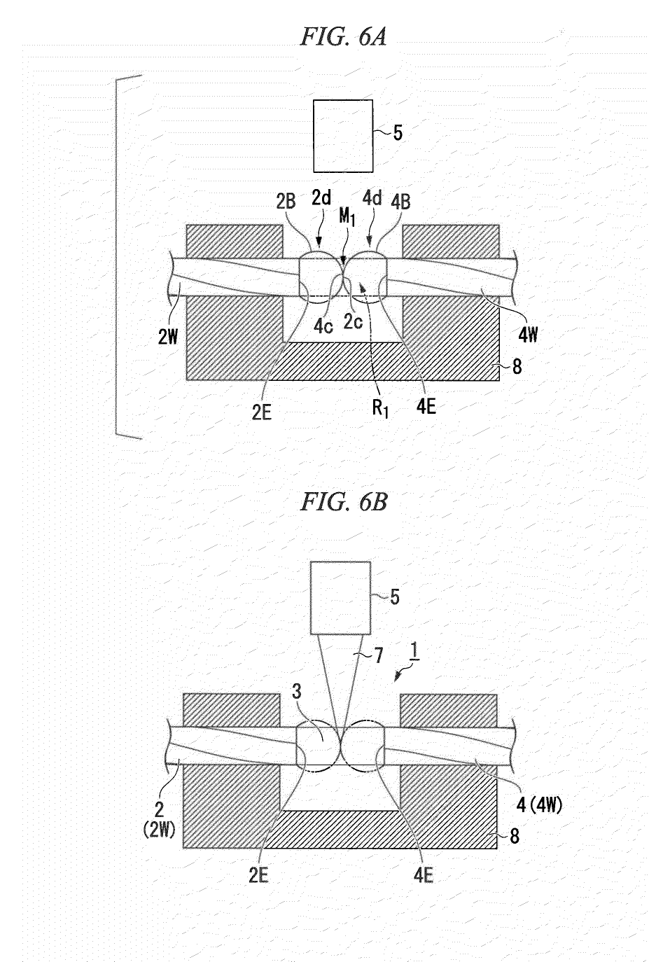

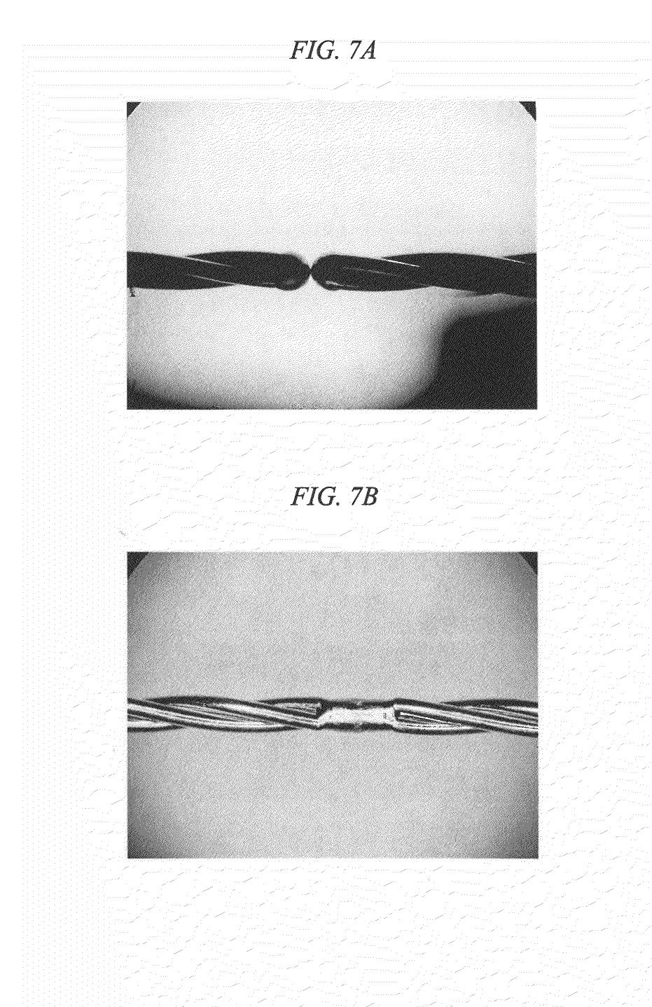

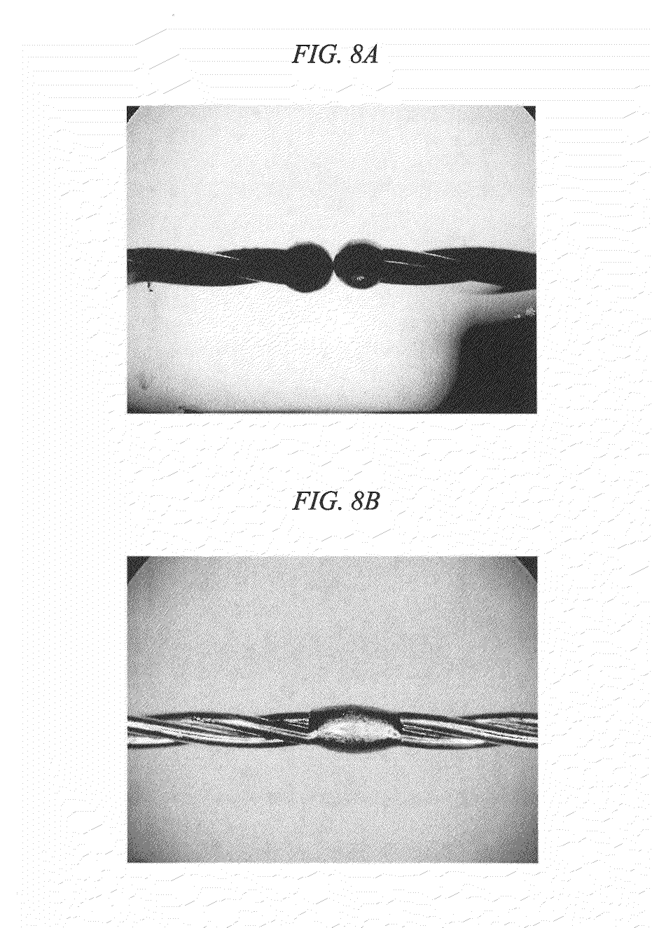

FIGS. 2A and 2B are schematic process explanatory drawings that illustrate a melting portion forming process of the production method of the medical wire according to the embodiment. FIG. 3 is a photographic image that illustrates an example of the melting portion formed in the melting portion forming process of the production method of the medical wire according to the embodiment. FIG. 4 is a graph that illustrates an example of a test result illustrating a relationship between a laser output and an outer diameter of the melting portion. A horizontal axis of the graph of FIG. 4 represents a laser output (W), and a vertical axis thereof represents an outer diameter (mm) of the melting portion. FIG. 5 is a photographic image that illustrates an example of an aspect of an end portion of the wire when the laser output is too low. FIGS. 6A and 6B are schematic process explanatory drawings that illustrate a joining process of the production method of the medical wire according to the embodiment. FIG. 7A is a photographic image that illustrates an example of the wire before joining in the joining process of the production method of the medical wire according to the embodiment. FIG. 7B is a photographic image that illustrates an example of the wire after joining in the joining process of the production method of the medical wire according to the embodiment. FIG. 8A is a photographic image that illustrates an example of the wire before joining in the joining process of a comparative example. FIG. 8B is a photographic image that illustrates an example of the wire after joining in the joining process of a comparative example.

In the production method of the medical wire of the embodiment, after the melting portion forming process is performed on non-joined stranded wires 2W and 4W (see FIG. 2A) having the same wire configuration as the stranded wire portions 2 and 4, respectively, the joining process thereof is performed.

First, in the melting portion forming process of the stranded wire 2W, as illustrated in FIG. 2A, the stranded wire 2W is held by a wire fixing jig 6. At this time, the stranded wire 2W is held so that the end portion 2A of the wire protrudes from the wire fixing jig 6 by a constant length h₁. In the embodiment, the wire end portion 2A is held so as to protrude in the vertical direction.

The length h₁ of the wire end portion 2A is set to be a length in which a spherical lump having a diameter d₂ slightly greater than the wire outer diameter d₁ of the stranded wire 2W is formed when the wire end portion 2A having the length h₁ is melted and solidified. The diameter d₂ may be set to a size in which the outer diameter of the fusion portion 3 formed in the joining process to be described below is less than or equal to the wire outer diameter d₁, and a suitable strength is obtained. When using the material of the embodiment, for example, the diameter d₂ is preferably within the range of 100% to 130% with respect to the wire outer diameter d₁.

Next, a laser irradiation device 5 is disposed above the end portion 2A of the wire. In the laser irradiation device 5, a suitable laser beam source having an output capable of heating and melting the end portion 2A of the wire can be adopted. In the embodiment, a laser beam source having a wavelength of 1070 nm, a maximum output of 60 W to 110 W, and a spot diameter of 20 μm to 40 μm may be adopted.

Next, as illustrated in FIG. 2B, the laser beam 7 is radiated to the end portion 2A of the wire from the laser irradiation device 5 disposed above the end portion 2A of the wire. As a result, the end portion 2A of the wire is heated, and the element wire 2a is melted to form a lump of liquid and deformed into a substantially spherical shape (including a strict spherical shape) due to surface tension.

The wire fixing jig 6 configured to hold the stranded wire 2W is able to maintain a solid state while the laser beam 7 is irradiated.

After the whole end portion 2A of the wire is melted, the irradiation of the laser beam 7 is stopped and radiationally cooled.

As a result, a massive melting portion 2B is formed in the wire end 2E formed on the upper end portion of the stranded wire 2W held by the wire fixing jig 6.

That is, the melting portion 2B is formed in an approximately spherical shape due to surface tension in the liquid state, and is solidified due to the radiational cooling while maintaining the shape thereof. In the embodiment, the shape of the melting portion 2B is formed in an approximately spherical shape of a diameter d₂. In addition, the diameter d₂ in a case in which the melting portion 2B is not a strict spherical shape means an average diameter in a direction perpendicular to the central axis of the wire.

A shape range of an approximately spherical shape is able to permit a range of variation of a shape occurred by a balance between surface tension and a gravitational force, shrinkage during solidification or the like.

For example, in a specific example of a 1×3 configuration of the stranded wire 2W of the embodiment, when relationships of d₀=0.25 (mm), d₁=0.52 (mm), and h₁=3 (mm) are satisfied, by performing the irradiation of the laser beam 7 of the laser output of 80 W with the pulse width of 100 (ms) by 1 pulse, the end portion 2A of the wire was melted. In this case, the diameter d₂ of the melting portion 2B during solidification satisfied a relationship of d₂=0.55 (mm).

The photographic image of the stranded wire 2W before joining formed at this time is illustrated in FIG. 3. It is understood that an approximately spherical melting portion is formed in the end portion.

Furthermore, when only the laser output of the laser beam 7 is changed under the same condition, the outer diameter d₂ of the melting portion 2B can be changed. As the specific example, the measurement result of the outer diameter d₂ of the melting portion 2B when the laser output is changed from 40 W to 180 W is represented in the graph of FIG. 4.

It is understood that when the laser output is from 60 W to 180 W, the outer diameter d₂ also increases with an increase of laser output. By performing such a test in advance, it is possible to obtain the laser output for obtaining a suitable outer diameter d₂.

In addition, when the laser output is 40 W, as illustrated in the photographic image of FIG. 5, since the approximately spherical melting portion 2B is not formed, the data of the outer diameter is also not plotted.

When the laser output is too low in this manner, since a melting quantity is too small, the approximately spherical melting portion 2B is not formed.

Next, the stranded wire 2W formed with the melting portion 2B is detached from the wire fixing jig 6, and as illustrated in FIG. 2A, in the same manner as described above, the stranded wire 4W is held on the wire fixing jig 6, instead of the stranded wire 2W. At this time, in the same manner as described above, a wire end portion 4A having a length h₁ is projected above the wire fixing jig 6 and the stranded wire 4W is held.

Next, as illustrated in FIG. 2B, in the same manner as described above, the end portion 4A of the wire is irradiated with the laser beam 7 to form the melting portion 4B.

After the melting portion 4B is solidified, the stranded wire 4W formed with the melting portion 4B in the wire end 4E is detached from the wire fixing jig 6.

In this way, the melting portion forming process is finished.

In this manner, the melting portion forming process is a process of forming massive melting portions 2B and 4B in each of the wire ends 2E and 4E by melting and solidifying the end portions 2A and 4A of the wires of the stranded wires 2W and 4W, respectively.

Next, the joining process is performed.

In this process, as illustrated in FIG. 6A, in a state in which the tip portions 2c and 4c of the melting portions 2B and 4B are caused to abut each other using a clamp jig 8, the stranded wires 2W and 4W are clamped at a coaxial position in a horizontal direction.

At this time, since vicinities of the tip portions 2c and 4c are formed to have a convex curved surface, the melting portions 2B and 4B come into point-contact with each other, and a groove portion M₁ interposed between the surfaces of the melting portions 2B and 4B is formed near the tip portions 2c and 4c.

Furthermore, since side portions 2d and 4d of the melting portions 2B and 4B are formed in an approximately spherical shape having a diameter d₂ greater than the wire outer diameter d₁, the side portions 2d and 4d protrude outward in the radial direction from a cylindrical area R₁ having a diameter d₁ that connects the end portions of the stranded wires 2W and 4W.

Next, the laser irradiation device 5 is disposed above an abutment position between the melting portions 2B and 4B, and as illustrated in FIG. 6B, the laser beam 7 is irradiated toward the abutment position. The laser beam 7 at this time has energy to an extent that the whole melting portions 2B and 4B can be melted. In a specific example of the embodiment, for example, an irradiation condition is provided in which the laser beam of 120 W is radiated by 1 pulse with a pulse width of 100 ms.

When the melting portions 2B and 4B start melting from the abutment portion due to the irradiation of the laser beam 7, surface tension acts on the molten portion, for example, the side portions 2d and 4d located outside the cylindrical area R₁ move to the groove portion M₁ side, and the molten portions tend to condense in a cylindrical shape.

For this reason, the molten portions of the melting portions 2B and 4B are fused with each other, and are deformed into a cylindrical rod shape connected to the end portions of the stranded wires 2W and 4W.

When the irradiation of the laser beam 7 is stopped, the molten portions of the melting portions 2B and 4B are solidified due to heat dissipation to form the fusion portion 3. As a result, the wire ends 2E and 4E of the stranded wires 2W and 4W are joined to each other via the fusion portion 3, and the joining wire 1 is produced.

Next, the joining wire 1 is detached by releasing the clamp of the clamp jig 8.

In this way, the joining process of the embodiment is finished.

In the joining process of the embodiment, the melting portions 2B and 4B are caused to abut each other to melt the melting portions 2B and 4B again, and the fusion portion 3 formed by the fusion of the melting portions 2B and 4B is formed and solidified, thereby joining the wire ends 2E and 4E via the fusion portion 3.

The fusion portion 3 is formed in a solid rod shape in which the melting portions 2B and 4B are alloyed by melting and fusing with each other. For this reason, a volume of the fusion portion 3 is approximately equal to the sum of the volume of the melting portions 2B and 4B before melting.

Examples of causes of errors may include a decrease in volume which is caused when the molten metal is penetrated through a wire gap between the wire ends 2E and 4E of the stranded wires 2W and 4W adjacent to the molten portion, the melting thereof starts from the wire ends 2E and 4E. These causes of error can be evaluated by performing the test in advance. For this reason, by setting the volume of the melting portions 2B and 4B, that is, the length h₁ of the end portions 2A and 4A, of the wire in the melting portion forming process to the suitable value, the volume of the fusion portion 3 can be controlled.

In the embodiment, it is possible to form the shape of the fusion portion 3 in an approximately cylindrical shape having a diameter of d=0.5 (mm) and a length of L₁=1 (mm) by a numerical example of h₁ mentioned above.

FIG. 7A illustrates a photographic image that illustrates a form before joining of the stranded wires 2W and 4W in the specific example. Furthermore, FIG. 7B illustrates a photographic image that illustrates a form after joining of the stranded wires 2W and 4W in the specific example.

It is understood that an approximately cylindrical joining portion is formed after joining.

In this manner, in the production method of the medical wire of the embodiment, the shape control of the fusion portion 3 can be easily performed by performing the melting portion forming process and the joining process in this order.

For example, since the stranded wires 2W and 4W are formed by the stranded wires, spatial homogeneity is poorer than that of the single line wire. For this reason, when the end portions of the stranded wires 2W and 4W are caused to abut each other to irradiate the laser beam 7 without forming the melting portions 2B and 4B, the method of melting may become non-uniform, and thus there is a concern that the end portions may be joined in a distorted shape.

Furthermore, the stranded wires 2W and 4W have a lower apparent density than the single wire having the same wire outer diameter. For this reason, there is a probability that it is difficult to obtain a sufficient quantity of melting required to join the wire ends, therefore a diameter becomes too thin, or the molten portion is separated due to surface tension.

According to the embodiment, in order to cause melting to be started from the abutment position between the approximately spherical melting portions 2B and 4B due to a solid metal, the melting progresses from the abutment position toward each of the melting portions 2B and 4B in a well-balanced manner. For this reason, the shape of the fusion portion 3 is formed in a cylindrical rod shape or a smooth rod-like shape with a thin intermediate portion according to surface tension. Furthermore, an error of the outer diameter of the fusion portion 3 can also be suppressed.

Particularly, as described above, since the causes of error act on a way in which the outer diameter of the fusion portion 3 becomes thinner, the outer diameter is difficult to increase.

However, when the outer diameters d₂ of the melting portions 2B and 4B are too great, since the volume after the solidification becomes greater than the volume of the cylinder area R₁, the outer diameter d of the fusion portion 3 becomes greater than the wire outer diameter d₁.

For example, FIGS. 8A and 8B illustrate photographic images of an example of a case in which the laser beam of the laser output of 120 W is irradiated by 1 pulse with the pulse width or 100 ms to form the melting portion and perform joining, as comparative examples. As illustrated in FIG. 8B, the outer diameter of the solidified fusion portion becomes greater than the wire outer diameter d₁.

In the above-mentioned specific example, the outer diameter d of the fusion portion 3 was less than or equal to the wire outer diameter d₁, and the range of the laser output in which satisfactory strength is obtained was 60 to 110 W.

In this manner, in the embodiment, a relationship between the outer diameter d₂ of the melting portions 2B and 4B and the outer diameter d of the fusion portion 3 is examined in advance, and for example, the working conditions such as the laser output are suitably set. As a result, since there is no need to correct an excess portion protruding from the wire outer diameter to the outside by secondary processing, the medical wire can be produced at a low cost.

Furthermore, since the fusion portion 3 melts the melting portions 2B and 4B that have already been melted again and fuses the melting portions 2B and 4B, the elements in the molten portion are melted uniformly and converted into an alloy. As a result, defects of the joining portion scarcely occur, and reliability of the joining wire 1 can be improved.

Furthermore, since the fusion portion 3 can be formed without using an another member such as a coupling member for joining, the production thereof is easy, the number of components can be reduced, and the production thereof can be made at a low cost.

### First Modified Example

Next, a medical wire of a first modified example of the first embodiment of the invention will be described.

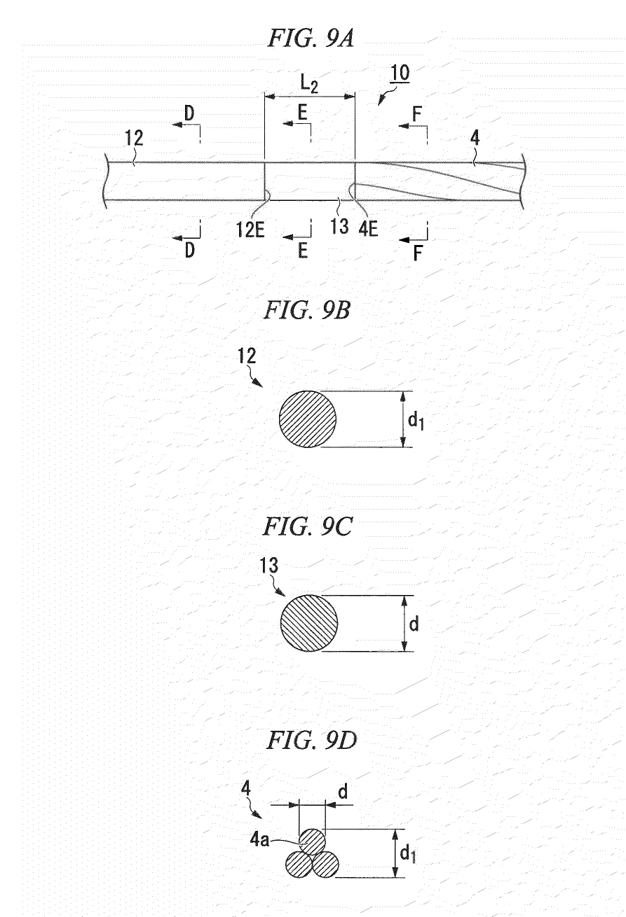

FIG. 9A is a schematic front view that illustrates a schematic configuration of the medical wire according to the modified example. FIG. 9B is a cross-sectional view taken along line D-D of FIG. 9A. FIG. 9C is a cross-sectional view taken along line E-E of FIG. 9A. FIG. 9D is a cross-sectional view taken along line F-F of FIG. 9A.

As illustrated in FIG. 9A, the joining wire 10 (medical wire) of the modified example includes a single line wire portion 12 and a fusion portion 13, instead of the stranded wire portion 2 and the fusion portion 3 of the joining wire 1 of the first embodiment.

The single line wire portion 12 is a single line wire made of stainless steel and having the diameter d₁.

The fusion portion 13 is a part in which the end portion of the single line wire portion 12 and the end portion of the stranded wire portion 4 are melted, fused, and solidified. In the modified example, as illustrated in FIG. 9C, the fusion portion is formed in a solid rod shape having a circular cross section with an outer diameter of d. An axial length of the fusion portion 3 is L₂ (see FIG. 9A). The axial end portions of the fusion portion 3 are connected to the wire ends 12E and 4E, respectively.

The joining wire 10 having such a configuration can be used as a manipulation rod or the like that performs the advance and retreat action of a catheter, a treatment tool or the like, by including the single line wire portion 12 in addition to the same application as the joining wire 1 of the above-mentioned embodiment.

Next, the production method of the joining wire 10 will be described.

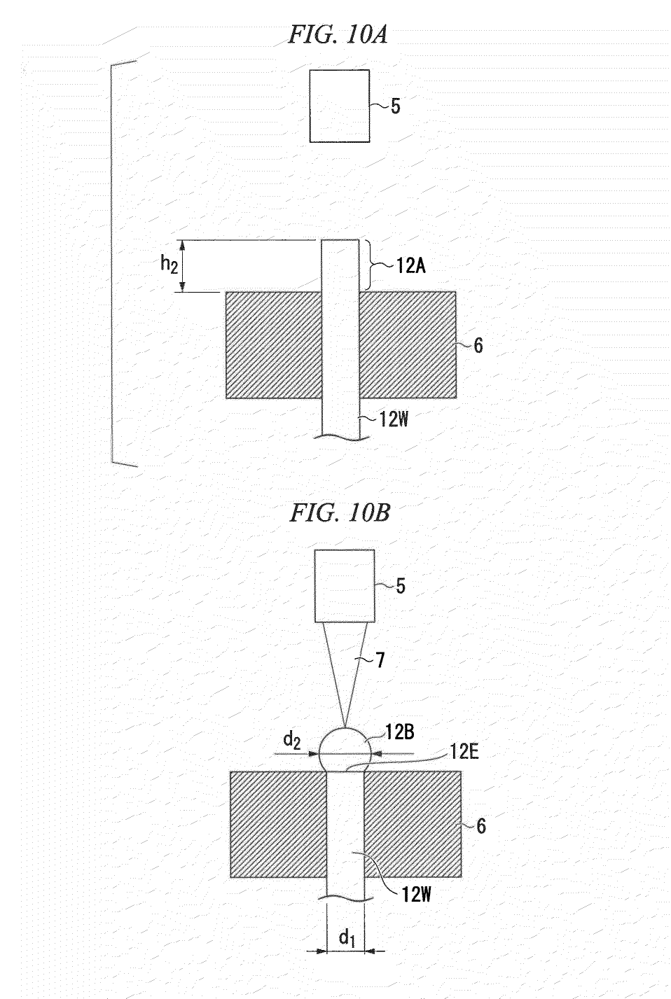

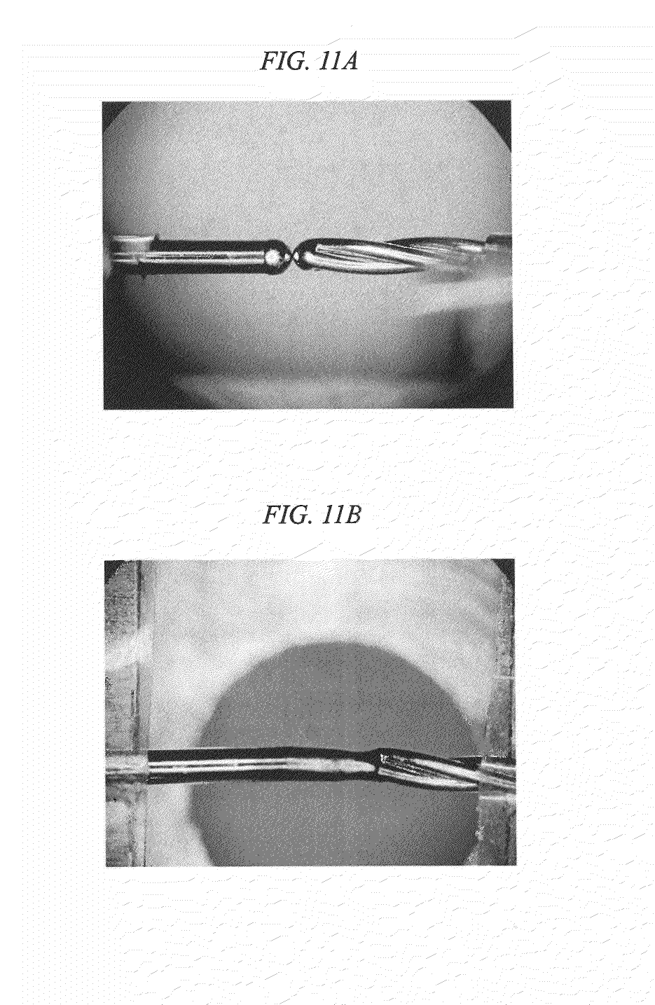

FIGS. 10A and 10B are schematic process explanatory drawings that illustrate a melting portion forming process of the production method of the medical wire according to the modified example. FIG. 11A is a photographic image that illustrates an example of the wire before in the joining process of the production method of the medical wire according to the modified example. FIG. 11B is a photographic image that illustrates an example of the wire after joining in the joining process of the production method of the medical wire according to the first modified example of the invention.

The joining wire 10 of the modified example is produced by performing approximately the same melting portion forming process as in the first embodiment on a non-joined single line wire 12W and the stranded wire 4W having the same wire configurations as the single line wire portion 12 and the stranded wire portion 4 (see FIG. 10A), and then performing the joining process thereof. Hereinafter, the points different from in the above-mentioned embodiment will be mainly described.

In the melting portion forming process of the single line wire 12W of the modified example, since the single line wire 12W is a solid member, as illustrated in FIG. 10A, the single line wire 12W is held by the wire fixing jig 6 so that the end portion 12A of the wire protrudes from the wire fixing jig 6 by a constant length h₂. For example, in the modified example, it is assumed that a relationship of h₂=3 (mm) is satisfied. By forming the melting portion 12B in the distal end of the single line wire 12W, since the distal end shape at the time of cutting the single lire wire 12W can be formed in the approximately spherical melting portion 12B, the condition of the joining process of the fusion portion at the subsequent stage can be stably performed.

When the end portion 12A of the wire is irradiated with the laser beam 7 in this state, as illustrated in FIG. 10B, the melting portion 12B having the diameter of d₂ is formed at the wire end 12E.

The joining process of the modified example is the same as the process of the above-mentioned embodiment except that the single line wire 12W formed with the melting portion 12B is used instead of the stranded wire 2W formed with the melting portion 2B of the above-mentioned embodiment.

In this manner, the joining wire 10 is produced.

The modified example is an example of a case in which the stranded wire and the single line wire are joined to each other to produce the medical wire.

FIG. 11A illustrates a photographic image that illustrates a form before joining of the single line wire 12W and the stranded wire 4W in the specific example. Furthermore, FIG. 11B illustrates a photographic image that illustrates a form after joining.

It is understood that the approximately cylindrical fusion portion is formed after joining.

### Second Modified Example

Next, a medical wire of a second modified example of the first embodiment of the invention will be described.

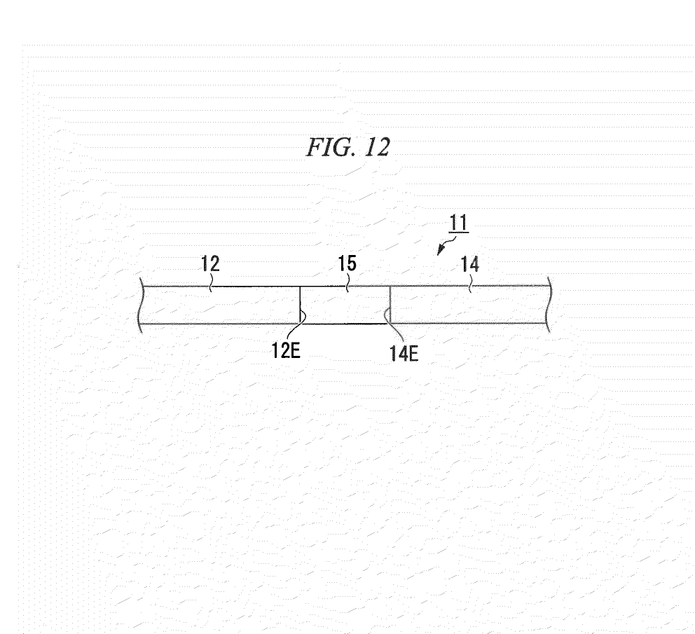

FIG. 12 is a schematic front view that illustrates a schematic configuration of the medical wire of the modified example.

As illustrated in FIG. 12, a joining wire H (medical wire) of the modified example includes a single line wire portion 14 and a fusion portion 15, instead of the stranded wire portion 4 and the fusion portion 13 of the joining wire 10 of the first modified example. Hereinafter, points different from the first modified example will be mainly described.

The single line wire portion 14 is a single line wire having a diameter of d₁.

The fusion portion 15 is formed in a solid rod shape having a circular cross section and having an outer diameter d similarly to the fusion portion 13, in a part in which the end portions of the single line wire portions 12 and 14 are melted, fused, and solidified. The axial end portions of the fusion portion 15 are connected to the wire ends 12E and 14E, respectively.

The joining wire 11 having such a configuration can be used in the same application as the first modified example.

The joining wire 11 can be produced by forming the melting portion in the wire end 14E in approximately the same manner as the single line wire 12W of the first modified example using a single line wire (not illustrated) having the same cross-sectional shape as the single line wire portion 14, and performing the same joining process as the first embodiment.

However, when the single line wires are joined, the outer diameter of a molting portion (not illustrated) formed on a distal end of a single line wire (not illustrated) for forming the single line wire portion 14 is set to, for example, an outer diameter that is smaller than the outer diameter d₂ of the melting portion 4B of the stranded wire 4W of the first modified example and is close to the outer diameter d₁ of the single line wire portion 14. As a result, it is possible to correct the volume since the decrease in volume due to the penetration of the molten metal in the wire gap specific to the stranded wire does not occur, and thus the outer diameter of the fusion portion 15 can be controlled.

The modified example is an example of a case in which the medical wire is produced by joining the single line wires to each other.

### Third Modified Example

Next, a medical wire of a third modified example of the first embodiment of the invention will be described.

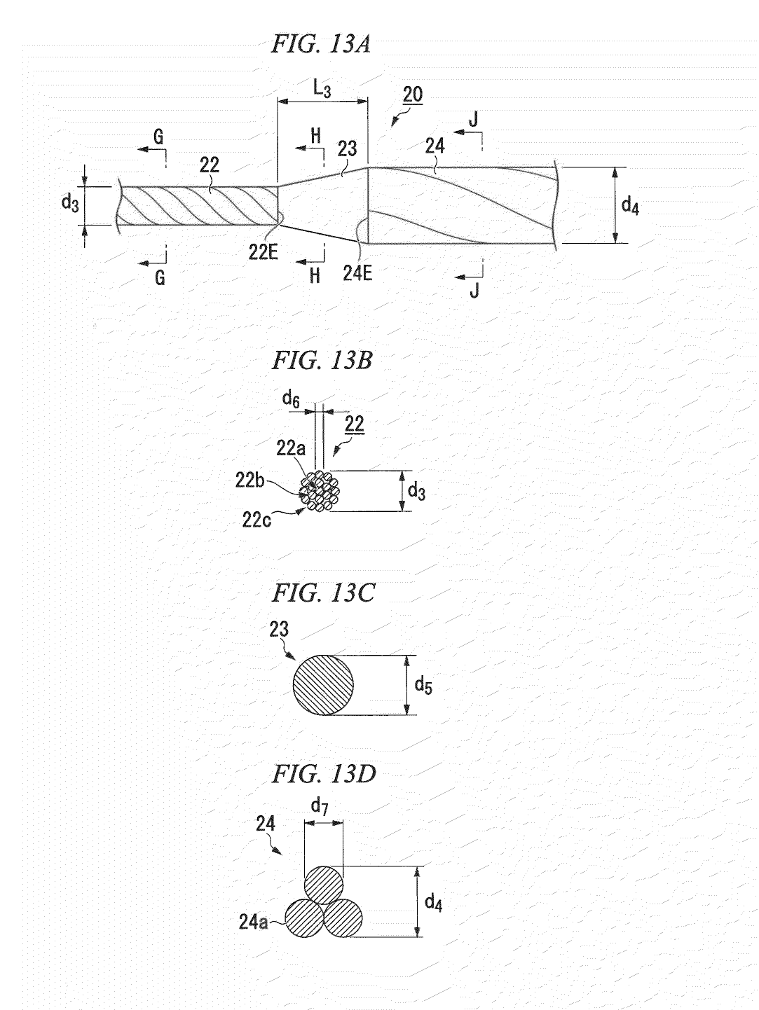

FIG. 13A is a schematic front view that illustrates a schematic configuration of a medical wire according to the modified example. FIG. 13B is a cross-sectional view taken along line G-G of FIG. 13A. FIG. 13C is a cross-sectional view taken along line H-H of FIG. 13A, FIG. 13D is a cross-sectional view taken along line J-J of FIG. 13A.

As illustrated in FIG. 13A, the joining wire 20 (medical wire) of the modified example includes stranded wire portions 22 and 24 (stranded wires), and a fusion portion 23, instead of the stranded wire portion 2, the stranded wire portion 4, and the fusion portion 3 of the joining wire 1 of the above-mentioned embodiment. Hereinafter, points different from the above-mentioned embodiment will be mainly described.

As illustrated in FIG. 13B, the stranded wire portion 22 has a wire configuration of 1×19 in which one core wire 22a, six element wires 22b, and twelve element wires 22c are twisted from the center toward the outer circumference.

Wire diameters of the core wire 22a and the element wires 22b and 22c forming the stranded wire portion 22 are all set to d₆=0.06 (mm) as an example, and as a result, a wire outer diameter d₃ of the stranded wire portion 22 is d₃=0.30 (mm).

Examples of a material of the core wire 22a and the element wires 22b and 22c of the stranded wire portion 22 may include stainless steel.

As illustrated in FIG. 13D, the stranded wire portion 24 has a wire configuration of 1×3.

The wire diameter of each element wire 24a forming the stranded wire portion 24 is set to, an example, d₇=0.25 (mm), and as a result, a wire outer diameter d₄ of the stranded wire portion 24 is d₄=0.52 (mm).

Examples of a material of each element wire of the stranded wire portion 24 may include stainless steel.

In this manner, the stranded wire portion 22 is a stranded wire that has higher flexibility and a smaller wire outer diameter than the stranded wire portion 24 due to twisting of the plurality of small-diameter element wires.

Furthermore, the stranded wire portion 24 is a stranded wire that has lower flexibility and a larger wire outer diameter than the stranded wire portion 22 due to twisting of a few large-diameter element wires.

The fusion portion 23 is a part in which the end portion of the stranded wire portion 22 and the end portion of the stranded wire portion 24 are melted, fused and solidified. In the modified example, as illustrated in FIG. 13A, the fusion portion 23 has a solid rod shape having a tapered shape in which the diameter gradually increases from d₃ to d₄ from the end portion of the stranded wire portion 22 toward the end portion of the stranded wire portion 24. For this reason, as illustrated in FIG. 13C, the cross section of the fusion portion 23 is formed in a circular shape having a diameter d₅ (here, d₃≦d₅≦d₄). As illustrated in FIG. 13A, the axial length of the fusion portion 23 is L₃. The axial end portions of the fusion portion 23 are connected to the wire ends 22E and 24E, respectively.

For this reason, the fusion portion 23 is formed in a rod shape and has an outer diameter that is less than or equal to the wire outer diameter of the stranded wire portion 24 having the maximum outer diameter of the stranded wire portions 22 and 24.

In addition, the tapered shape of the fusion portion 23 may be a truncated cone shape having a constant slope, and the outer diameter of the truncated cone shape may change so that an axial intermediate portion becomes thinner. Furthermore, the cross-sectional shape may also be an elliptical shape, without being limited to a strict circular shape.

The joining wire 20 having such a configuration can be used in the same application as the joining wire 1 of the above-mentioned embodiment. Particularly, the joining wire 20 has stranded wire portions 22 and 24 having different flexibility and wire outer diameters from each other. The joining wire 20 is particularly suitable for the application of performing more delicate treatments using an endoscope treatment tool when the wire outer diameter of the stranded wire of the distal end portion is formed to be thinner than the wire outer diameter of the stranded wire of the long portion.

Next, a production method of the joining wire 20 will be described.

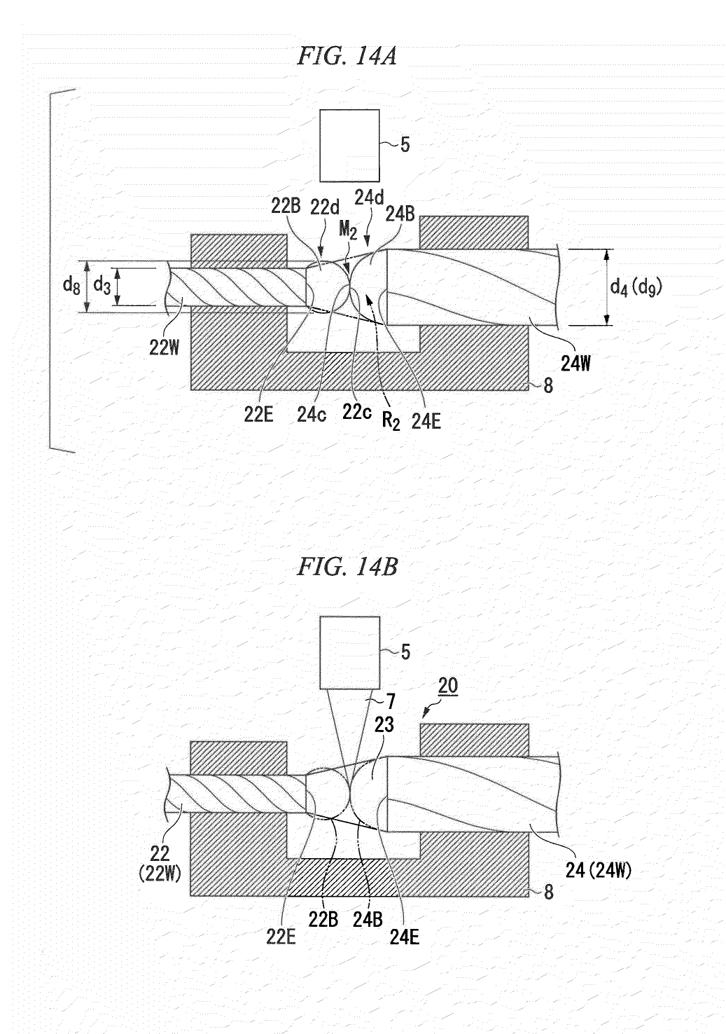

FIGS. 14A and 14B are schematic process explanatory drawings that illustrate the joining process of the production method of the medical wire according to a third modified example of the first embodiment of the invention.

The joining wire 20 of the modified example is produced by performing approximately the same melting portion forming process as in the first embodiment on non-joined stranded wires 22W and 24W (see FIG. 14A) having the same wire configuration as the stranded wire portions 22 and 24, and then performing the joining process. Hereinafter, points different from the first embodiment will be mainly described.

In the melting portion forming process of the stranded wires 22W and 24W of the modified example, since the outer diameters are different form the stranded wires 2W and 4W of the first embodiment, the outer diameters of the melting portions 22B and 24B formed at the wire ends 22E and 24E of the stranded wires 22W and 24W are different from each other. In this case, an outer diameter d₈ of an approximately spherical melting portion 22B of the stranded wire 22W is greater than the wire outer diameter d₃ of the stranded wire 22W. In contrast, an outer diameter d₉ of the melting portion 24B of an approximately spherical melting portion 24B of the stranded wire 24W has the same size as the wire outer diameter d₄ of the stranded wire 24W. That is, the outer diameter d₉ of the melting portion 24B is equal to the wire outer diameter d₄, and the melting portion 24B is formed in a hemispherical shape.

This is an example of a condition for preventing the maximum outer diameter of the melting portion 24B from exceeding the outer diameter d₄ of the stranded wire 24W. When the outer diameter d₃ of the stranded wire 22W is thinner, there is a need to further reduce the volume of the melting portion 24B.

In this case, the melting portion 24B may be formed in a shape of a partially spherical body protruding from the wire end 24E within the range in which the diameter does not exceed a circle having a diameter of d₄. That is, the melting portion 24B may have a greater radius of curvature than d₄, and may be formed in a shape of a partially spherical body that does not protrude outward in the radial direction of the stranded wire 24W in the wire end 24E.

In the modified example, the respective outer diameters d₈ and d₉ are set to d₈=0.3 (mm) and d₉=0.6 (mm) that are equivalent to 120% and 100% with respect to the corresponding wire outer diameters d₃ and d₄.

In order to form the melting portions 22B and 24B having such a shape, a protrusion length from the wire fixing jig 6 may be suitably adjusted, and the irradiation condition of the laser beam 7 may be set in accordance with the melting volume.

As the diameters d₈ and d₉ of the melting portions 22B and 24B, sizes may be set in which the fusion portion 23 formed in the joining process to be described below is formed in a shape entering a truncated cone area R₂ (see FIG. 14A) having the outer diameters changed from d₃ to d₄ in the length L₃, and suitable strength is obtained.

The joining process of the modified example uses the stranded wire 22W formed with the melting portion 22B instead of the stranded wire 2W formed with the melting portion 2B of the first embodiment, and uses the stranded wire 24W formed with the melting portion 24B instead of the stranded wire 4W formed with the melting portion 4B. The joining process is approximately the same as that of the first embodiment other than this point.

In the process, as illustrated in FIG. 14A, in a state in which the respective tip portions 22c and 24c of the melting portions 22B and 24B are caused to abut each other using the clamp jig 8, the stranded wires 22W and 24W are clamped at the coaxial position in the horizontal direction.

At this time, since vicinities of the tip portions 22c and 24c are formed to have a convex curved surface, the melting portions 22B and 24B come into point-contact with each other, and a groove portion M₂ interposed between the surfaces of the melting portions 22B and 24B is formed near the tip portions 22c and 24c.

Herein, since the side portion 22d of the melting portion 22B is formed in an approximately spherical shape and has a diameter d₈ greater than the wire outer diameter d₃, the side portion 22d protrudes outward in the radial direction from the truncated cone area R₂ that connects the end portions of the stranded wires 22W and 24W. Meanwhile, the side portion 24d of the melting portion 24B is located inside the truncated cone area R₂.

Next, the laser irradiation device 5 is disposed above the abutment position between the melting portions 22B and 24B, and as illustrated in FIG. 14B, the laser beam 7 is irradiated toward the abutment position. At this time, the laser beam 7 has energy of an extent capable of melting the entire melting portions 22B and 24B.

When the melting portions 22B and 24B start melting from the mutual abutment portion due to the irradiation of the laser beam 7, surface tension acts on the molten portion, for example, the side portion 22d located outside the truncated cone area R₂ moves to the groove portion M₂ side, and the molten portion tends to aggregate in a truncated cone-shape. Meanwhile, since the side portion 24d of the melting portion 24B is located inside the truncated cone area R₂ before melting, when melting starts, the side portion 24d is pulled toward the groove portion M₂ due to surface tension, and the side portion 24d does not widen to the outer side of the truncated cone area R₂.

For this reason, the molten portions of the melting portions 22B and 24B are fused with each other and are deformed into a truncated conical rod shape that connects the end portions of the stranded wires 22W and 24W.

When the irradiation of the laser beam 7 is stopped, the molten portions of the melting portions 22B and 24B are solidified due to heat dissipation, and the fusion portion 23 is formed. As a result, the wire ends 22E and 24E of the stranded wires 22W and 24W are joined to each other via the fusion portion 23, and the joining wire 20 is produced.

Next, by releasing the clamp of the clamp jig 8, the joining wire 1 is detached.

Here, the joining process of the modified example is finished.

The modified example is an example in which the stranded wires having the different wire outer diameters are joined to each other to produce a medical wire.

When the wires having the different wire outer diameters are joined to each other using a coupling member or the like, a joining portion having an outer diameter greater than the greater wire outer diameter can be obtained. However, according to the Modified example, since such coupling member is not required, the wires can be joined by the fusion portion 23 having a smooth change of the cross-sectional shape without grinding an excess portion,

### Fourth to Sixth Modified Examples

Next, medical wires of fourth to sixth modified examples of the first embodiment of the invention will be described.

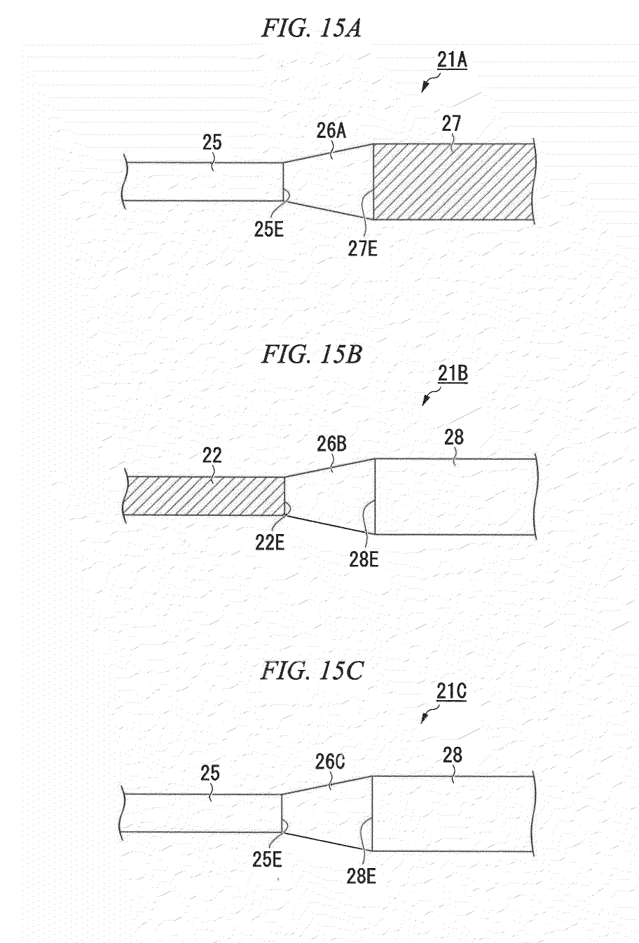

FIGS. 15A, 15B and 15C are schematic front views that illustrate schematic configurations of medical wires according to fourth; fifth and sixth modified examples of the first embodiment of the invention, respectively.

The fourth to sixth modified examples are modified examples in which the combination of the wires is changed in the medical wire in which the wires having the different wire outer diameters are connected as in the third modified example. Since it is clear that the medical wires can be produced in approximately the same manner as in the third modified example, only each configuration will be briefly described.

As illustrated in FIG. 15A, the joining wire 21A (medical wire) of the fourth modified example is an example in which a wire end 25E of a single line wire portion 25 (first wire) of a small diameter and a wire end 27E of a stranded wire portion 27 (second wire) of a large diameter are joined to each other by a tapered fusion portion 26A.

As illustrated in FIG. 15B, the joining wire 21B (medical wire) of the fifth modified example is an example in which the wire end 22E of the stranded wire portion 22 (first wire) of a small diameter and a wire end 28E of a single wire portion 28 (second wire) of a large diameter are joined to each other by a tapered fusion portion 26B.

As illustrated in FIG. 15C, the joining wire 21C (medical wire) of the sixth modified example is an example in which a wire end 25E of a single line wire portion 25 (first wire) of a small diameter and the wire end 28E of the single wire portion 28 (second wire) of a large diameter are joined to each other by a tapered fusion portion 26C.

### Seventh Modified Example

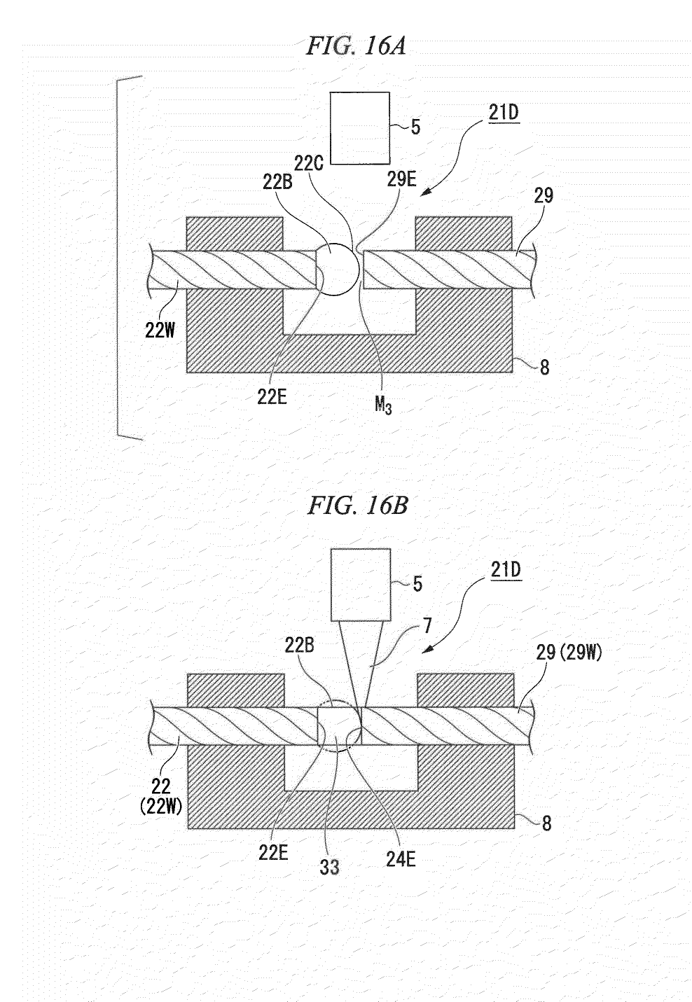

As illustrated in FIGS. 16A and 16B, a joining wire 21D (medical wire) of a seventh modified example is produced by performing substantially the same melting portion forming process as in the first embodiment on one wire end 2E of the stranded wires 22W and 24W (see FIG. 14A) having the same wire configurations as the stranded wire portions 22 and 24 to form the melting portion 2B and then performing the joining process. That is, the seventh modified example is an example in which one wire end 2E formed with the melting portion 2B is joined with the other wire end 4E (joined part) not having the melting portion. Hereinafter, points different from the first embodiment will be mainly described.

FIGS. 16A and 16B are schematic process explanatory drawings that describe the joining process of the production method of the medical wire according to the seventh modified example of the first embodiment.

In the joining process of the modified example, the stranded wire 22W (first wire) formed with the melting portion 22B is used instead of the stranded wire 2W formed with the melting portion 2B of the first embodiment. Furthermore, a stranded wire 29 (second wire) that is not formed with the melting portion is used instead of the stranded wire 4W formed with the melting portion 4B.

As illustrated in FIG. 16A, in the process, in a state in which a tip portion 22c of the melting portion 22B and a wire end 29E of the stranded wire 29 serving as the joined part are caused to abut each other, the stranded wires 22W and 29 are clamped at the coaxial position in the horizontal direction using the clamp jig 8.

At this time, since the vicinity of the tip portion 22c is formed on a convex curved surface, the melting portion 22B comes into point-contact with the wire end 29E of the stranded wire 29, and a groove portion M₃ is formed near the tip portion 22c.

Next, the laser irradiation device 5 is disposed above an abutment position between the melting portion 22B and the wire end 29E, and as illustrated in FIG. 16B, the laser beam 7 is irradiated toward the abutment position. Here, the laser beam 7 of this time has energy to an extent capable of melting the entire melting portion 22B.

When the melting portion 22B and the wire end 29B start melting from the abutment portion due to the irradiation of the laser beam 7, surface tension acts on the molten portion, and the melting portion tends to aggregate in a cylindrical shape. For this reason, the molten portions between the melting portion 22B and the wire end 29E are fused with each other, and are deformed to a cylindrical rod shape that is connected to the end portions of the stranded wires 22W and 29.

When the irradiation of the laser beam 7 is stopped, the molten portion between the melting portion 22B and the wire end 29E is solidified due to heat dissipation, and the fusion portion 33 is formed. As a result, the wire ends 22E and 29E of the stranded wires 22W and 29 are joined to each other via the fusion portion 33, and thus the joining wire 21D is produced.

Next, by releasing the clamp of the clamp jig 8, the joining wire 21D is detached. Here, the joining process of the embodiment is finished.

In addition, in the above-mentioned description, although a case in which two wires (the first wire and the second wire) facing each other are joined by the fusion portion has been described, the melting portions may be formed on both end portions of one wire (the first wire), and the fusion portion may be formed by each melting portion to form a loop-shaped medical wire.

Furthermore, in the above-mentioned description, an example of a case in which the outer diameter of the fusion portion is less than or equal to the wire outer diameter has been described. However, the shape of the fusion portion is formed based on the action of surface tension. For this reason, even when a fusion portion having an outer diameter greater than the wire outer diameter is formed, a shape is obtained in which the surface of the fusion portion is smooth, and the wire end is also smoothly connected. For this reason, as long as the outer diameter of the fusion portion is an outer diameter that is permissible in use, the outer diameter of the fusion portion may be greater than the wire outer diameter.

Furthermore, in the above-mentioned description, an example of a case in which the outer diameter of at least one of two melting portions forming the fusion portion is greater than the wire outer diameter formed with the melting portion has been described. However, when the outer diameter of the fusion portion can be form to have a desired size, the melting portion may set to have a shape in which neither side portion of both of the two melting portions protrudes from the range of the wire outer diameter.

Accordingly, as long as the melting portion is formed in a convex shape having an approximately spherical surface on the wire end, a radius of curvature of the melting portion surface can be set to a suitable size.

Furthermore, in the second modified example, an example of a case in which, when the single line wires are joined to each other, the outer diameter d₂ of the melting portion 12B of the single line wire 12W is greater than the wire outer diameter d₁, and the outer diameter of the melting portion of a single line wire (not illustrated) forming the single line wire portion 14 is smaller than the outer diameter d₂ but is greater than the wire outer diameter d₁ has been described. However, a magnitude correlation of the melting portion may be the reverse thereof. In all cases, the outer diameter of the melting portion may be set to be smaller than the outer diameter d₂.

Furthermore, in the above-mentioned description, although an example of a case in which the two wire ends facing each other are joined by the fusion portion has been described, as long as the number of the wire ends joined by the fusion portion is two more, the number is not particularly limited thereto. For example, plural wires having the melting portions may be arranged in parallel, and one or more wires formed with the melting portion may be caused to face the plural melting portions, thereby connecting multiple wires and one wire, or connecting multiple wires and multiple wires.

### Second Embodiment

Next, a medical wire according to the second embodiment will be described. In addition, in the following description and the drawings used in the description, the same components as the components already has been described are denoted by the same reference numerals, and the repeated description thereof will be omitted.

As illustrated in FIG. 1A, in the medical wire according to the first embodiment, two wire ends 2E and 4E are joined to each other, and the two wires ends 2E and 4E are joined to each other via the fusion portion 3. In contrast, as illustrated in FIGS. 18A and 18B, in the medical wire according to the second embodiment, the melting portion 2B is formed in one wire end 2E, and the melting portion 2B is caused to abut the joined part 16 serving as a certain position of the wire to form the fusion portion 36. Moreover, the wire end 2E and the joined part 16 are joined to each other via the fusion portion 36. In this respect, the embodiment differs from the first embodiment.

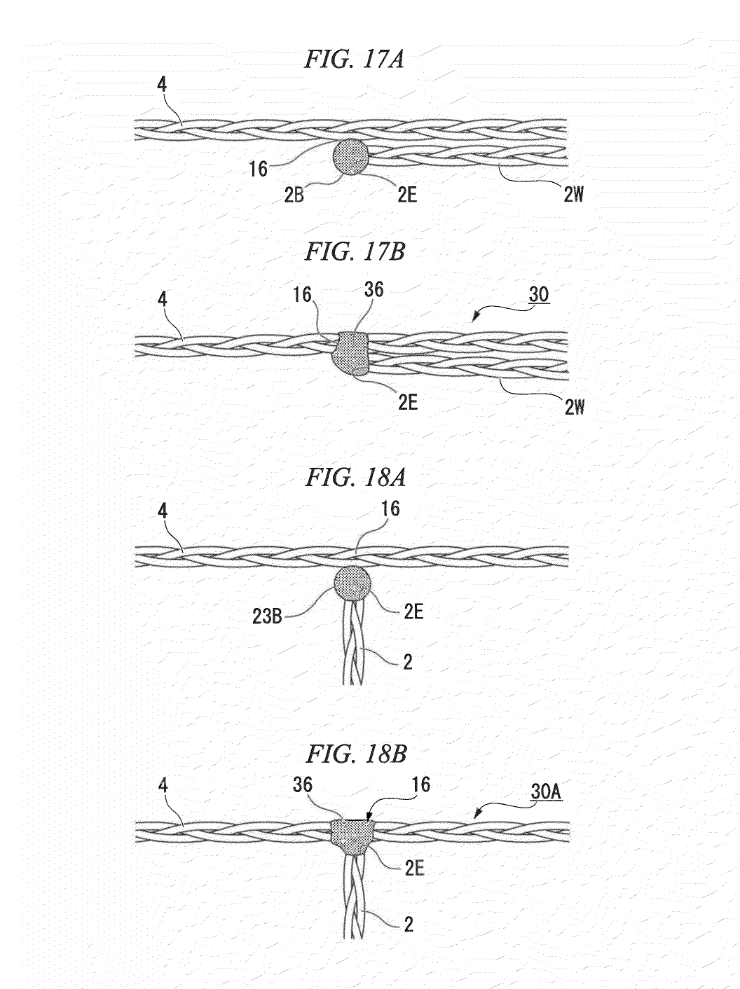

FIG. 17A is a front view that illustrates an example of the wire before joining in the joining process of the production method of the medical wire according to the embodiment. FIG. 17B is a front view that illustrates an example of the wire after joining in the joining process of the production method of the medical wire according to the embodiment. In the production method of the medical wire according to the embodiment, in the same manner as the process illustrated in FIGS. 2A and 2B, the Melting portion forming process is performed on the non-joined stranded wire 2W (the first wire) having the same wire configuration as the stranded wire portion 2.

Next, the joining process is performed. The melting portion 2B is caused to abut the joined part 16 serving as the outer circumferential portion of the stranded wire portion 4. Thereafter, in the same manner as in the first embodiment, the laser irradiation device 5 is disposed above the abutment position between the melting portion 2B and the joined part 16, and the laser beam 7 is radiated toward the abutment position, to perform the joining process.

When the irradiation of the laser beam 7 is stopped, the molten portion between the melting portion 2B and the joined part 16 is solidified due to heat dissipation; and the fusion portion 36 is formed. As a result, the wire end 2E of the stranded wire 2W and the joined part 16 are joined to each other via the fusion portion 36, and the joining wire 30 is produced.

### Eighth Modified Example

Next, a medical wire of a modified example (eighth modified example) of the second embodiment of the invention will be described.

The modified example is an example in which the wire end 2E formed with the melting portion 2B is joined approximately perpendicularly with respect to a second wire 4 having the joined part 16. The melting portion 23B is formed on the wire end 2E of a first wire 2 by the same method as in the first embodiment. The wire end 2E of the first wire 2 is caused to abut the joined part 16 serving as the outer circumferential surface of a certain position of the second wire 4. At this time, the first wire 2 is caused to abut the second wire 4 so that the longitudinal direction of the first wire 2 is approximately perpendicular to the longitudinal direction of the second wire 4, Moreover, the fusion portion 36 is formed by the same method as in the first embodiment to form the medical wire in which the first wire 2 and the second wire 4 are vertically joined to each other.

In addition, in the above-mentioned description, an example of a case in which two wires 2 and 4 (the first wire and the second wire) are joined to each other by the fusion portion 36 has been described. However; the melting portion 2B may be formed on the wire end 2E of the first wire 2, the outer circumferential portion of a certain position of the same wire as the first wire 2 may be used as the joined part 16, and the fusion portion 36 may be formed by the melting portion 2B and the joined part 16 to form a loop-shaped medical wire.

Furthermore, a connection angle between the first wire 2 having the melting portion 2B and the second wire 4 having the joined part 16 is not limited to an example illustrated in the second embodiment and the eighth modified example, and can be suitably set.

Furthermore, the entire components described in the above-mentioned embodiments, and various modified examples can be carried out by suitably changing or deleting the combination within the scope of the technical idea of the invention.

While preferred embodiments of the present invention have been described, the present invention is not limited to the embodiments. Additions, omissions, substitutions, and other variations may be made to the present invention within the scope that does not depart from the spirit of the present invention. The present invention is not limited by the above description, but only by the appended claims.

What is claimed is:

1. A production method of a medical wire, the method comprising:
a melting portion forming process that forms an massive melting portion in a wire end by melting and solidifying the wire end of a wire; and
a joining process that contacts the melting portion with a joined part, and melts and solidifies the melting portion and the joined part to form a fusion portion, thereby joining the wire end and a joined part via a fusion portion.

2. The production method of a medical wire according to claim 1,
wherein the wire includes a stranded wire.

3. The production method of a medical wire according to claim 1,
wherein the melting portion is formed in a substantially spherical shape in the wire end.

4. The production method of a medical wire according to claim 2,
wherein the melting portion is formed in a substantially spherical shape in the wire end.

5. The production method of a medical wire according to claim 1,
wherein the melting portion is formed by irradiating the wire end and the joined part with a laser beam.

6. The production method of a medical wire according to claim 1,
wherein the melting portion is formed in a first wire, and
the first wire or a second wire different from the first wire has the joined part.

7. The production method of a medical wire according to claim 6,
wherein the joined part is a wire end different from the wire end formed with the melting portion of the first wire or the wire end of the second wire.

8. The production method of a medical wire according to claim 7,
wherein the fusion portion is formed in a rod shape and has an outer diameter that is less than or equal to the maximum outer diameter of outer diameters of the first wire and the second wire.

9. The production method of a medical wire according to claim 6,
wherein the joined part is an outer circumferential portion of a certain position of one of the first wire and the second wire.

10. A medical wire comprising a fusion portion in which materials of a wire end of a wire and a joined part are melted and fused, the fusion portion being formed and joined between the wire end and the joined part that is to be joined with the wire end.

11. The medical wire according to claim 10,
wherein the wire includes a stranded wire.

12. The medical wire according to claim 10,
wherein the joined part is a wire end that is different from the wire end formed with the melting portion of the wire.

13. The medical wire according to claim 11,
wherein the joined part is a wire end that is different from the wire end formed with the melting portion of the wire.

14. The medical wire according to claim 12,
wherein the fusion portion is formed in a rod shape having an outer diameter that is less than or equal to the maximum outer diameter of outer diameters of the wire.

15. The medical wire according to claim 13,
wherein the fusion portion is formed in a rod shape having an outer diameter, that is less than or equal to the maximum outer diameter of outer diameters of the wires.

16. The medical wire according to claim 10,
wherein the joined part is an outer circumferential portion of the wire.

17. The medical wire according to claim 11,
wherein the joined part is an outer circumferential portion of the wire.
